(19) United States
(12) Patent Application Publication
(10) Pub. No.: US 2010/0303888 A1
(43) Pub. Date: Dec. 2, 2010

(54) COMPOSITION FOR ENHANCING BONE FORMATION

(76) Inventors: Jake Barralet, Montreal (CA); Damian Le Nihouannen, Montreal (CA); Svetlana Komarova, Kirkland (CA)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/450,949

(22) PCT Filed: Apr. 18, 2008
(86) PCT No.: PCT/CA2008/000733

§ 371 Date: Jan. 19, 2010

Related U.S. Application Data
(60) Provisional application No. 60/907,802, filed on Apr. 18, 2007.

(51) Int. Cl. A61K 9/00; A61K 38/18; C07C 59/19; A61P 39/06; A61P 41/00; A61P 19/08; C12N 5/07

(52) U.S. Cl. 424/425; 514/7.6; 424/484; 562/577; 435/377

(57) ABSTRACT

Disclosed herein is a matrix for inducing or enhancing osteoclast differentiation. The matrix comprises a material having an osteoclastogenic agent associated therewith, the agent being releasable from the material in an amount which is sufficient to induce or enhance osteoclast differentiation.

### FIELD OF THE INVENTION

The present invention concerns compositions for enhancing bone formation, bone and/or bone graft remodelling and more particularly to compositions for causing osteoclast differentiation in vitro or in vivo.

### BACKGROUND OF THE INVENTION

Current clinical approaches for the treatment of osteoporosis are based on inhibition of osteoclast action using, for example, bisphosphonates such as Fosamax or Boniva, or hormone replacement therapy, which at best preserve bone volume. Furthermore, long term use of the aforesaid treatment is thought to have, in some cases, resulted in adverse effects such as osteonecrosis and delayed fracture healing.

Various preclinical studies with bone morphogenic proteins (BMP), which are inductive factors, have shown that bone may be induced, however their use is limited. During the bone turnover process, bone formation is coupled to bone resorption, although molecular mediators of this process have not been identified. It was previously suggested that factors released from bone by resorbing osteoclasts might be responsible for the coupling. In addition, there is evidence which suggests that the osteoclasts may release soluble factors, which then affect osteoblasts.

It was recently demonstrated that a key osteoclastogenic factor Receptor Activator for Nuclear Factor κ B Ligand (RANKL) together with angiogenic factor VEGF (vascular endothelial cell growth factor) are required for efficient remodelling of devitalized autograft (Ito H, Koefoed M, Tiyapatanaputi P, at al. Remodeling of cortical bone allografts mediated by adherent rAAV-RANKL and VEGF gene therapy. Nature Medicine 11(3): 291-297(2005)). Deficiency in RANKL has also been shown to prevent allograft healing, suggesting the important role of RANKL in fracture healing.

Calcium phosphate cements are well known and have been used for numerous orthopedic and dental applications. For various reasons, such as impaired healing due to a diseased state (e.g. diabetes), micromovement, too large a defect, non physiological loading patterns, and age, bone healing in and around a graft or prosthesis may not proceed fully or at all. This often leads to additional surgeries and additional associated costs. Several attempts have been made by others to produce cements for use as implants to stimulate bone growth. Constantz at al. in United States patent application no. 2005/0106260 A1, published on May 19, 2005 for: “Calcium phosphate cements comprising an osteoclastogenic agent” disclose methods of producing flowable or paste compositions using calcium phosphate including RANKL and a setting agent. Zheng et al. in United States patent application no. 2003/0144197 A1, published Jul. 31, 2003 for: Autologous growth factor cocktail composition, method of production and use” disclose at least one extracted growth factor, including RANKL, suitable for treating osteogenesis or tenogenesis in a cement or calcium phosphate composition. Disadvantageously, RANKL's stability is unknown and it is thought to be unstable, requiring storage at −80° C., and it is typically added to culture medium twice in order to induce osteoclastogenesis. (“The calcium sensing receptor is directly involved in both osteoclast differentiation and apoptosis” Mentaverri R (Mentaverri, R.), Yano S (Yano, S.), Chattopadhyay N (Chattopadhyay, N.), Petit L (Petit, L.), Kifor O (Kifor, O.), Kamel S (Kamel, S.), Terwilliger E F (Terwilliger, E. F.), Brazier M (Brazier, M.), Brown E M (Brown, E. M.) FASEB 20 14 2562-+2006).

Thus there is a need for an improved composition, which is stable and which can be used to enhance bone formation.

### SUMMARY OF THE INVENTION

We have made the unexpected discovery that a matrix made from certain biomaterials, when loaded with osteoclastogenic agents (pro-osteoclastogenesis molecules), can induce osteoclastogenesis for at least up to 38 days when osteoclast precursors are within close proximity to the matrix. Advantageously, this will provide long term release of the osteoclastogenic agents from a variety of biomaterial matrices to enhance bone remodeling. This will lead to improved bone healing after surgery, accelerated implant fixation by bone ingrowth. Moreover, the matrices will provide simpler and less expensive methods to induce osteoclast differentiation in vitro, when compared to currently available methods. We have also unexpectedly discovered that the use of RANKL in combination with either sodium pyruvate or ascorbic acid with or without a matrix can enhance the osteoclastogenesis activity of RANKL

Accordingly, in one embodiment of the present invention there is provided a matrix for inducing or enhancing osteoclast differentiation, the matrix comprising: a material having an osteoclastogenic agent associated therewith, the agent being releasable from the material in an amount which is sufficient to induce or enhance osteoclast differentiation.

Typically, the material is a biomaterial. The material is microcrystalline. The material comprises crystals less than 500 μm in linear dimension. The material is porous. The material is porous at greater than 5% relative porosity. The material has a surface area of greater than 0.5 m² g⁻¹. The material is a ceramic or a non-metallic coating. The material is a calcium phosphate-containing cement. The material is a cement reactant or a mixture of cement reactants. The material is a cement reactant in a slurry. The cement is a cement product formed by the reaction of the cement reactants. The material is a diphosphate, or a triphosphate, or a tetraphosphate or a polyphosphate. The material is hydroxyapatite. The material is brushite. The material is a hydrogel. In one example, the osteoclastogenic agent is adsorbed onto the surface of the material at a concentration of 1 ng or more and 1000000 ng or less of agent per mg of material. The osteoclastogenic agent is adsorbed onto the surface of the material at a concentration of 10 ng or more and 500 ng or less of agent per mg of material. The osteoclastogenic agent is adsorbed onto the surface of the biomaterial at a concentration of 15 ng or more and 25 ng or less of agent per mg of material. In one example, the osteoclastogenic agent is RANKL. The RANKL is in combination with an antioxidant. The RANKL is in combination with an antioxidant and brushite. The RANKL is in combination with an antioxidant and hydroxyapatite. The antioxidant is ascorbic acid or dehydroascorbic acid or salts thereof. The ascorbic acid is L-ascorbic acid, D-ascorbic acid or DL-ascorbic acid, or salts thereof. The RANKL is in combination with pyruvate salts or pyruvic acid. The ascorbic acid is in combination with pyruvate salts or pyruvic acid. The ascorbic acid and the RANKL are located separately in the matrix and released simultaneously therefrom. The pyruvate salts or pyruvic acid and the RANKL are located separately in the matrix and released simultaneously therefrom. The RANKL is stable at ambient temperature. The RANKL is stable at 37° C. The osteoclastogenic agent is dried on the surface of the matrix. The osteoclastogenic agent is in an amount sufficient to cause or enhance implant osteointegration. The osteoclastogenic agent is in an amount sufficient to cause or enhance fractured bone to remodel. RANKL is in combination with pyruvate salts, pyruvic acid, ascorbic acid, or trehalose.

Accordingly, in another embodiment of the present invention, there is provided a composition for promoting osteoclastogenesis in vitro or in vivo, the composition comprising: a combination of RANKL and an antioxidant in amounts sufficient to promote osteoclastogenesis in vitro or in vivo.

Typically, the ratio of the RANKL to the antioxidant is 0.0001 to 100000. The antioxidant is ascorbic acid or dehydroascorbic acid or salts thereof. The ascorbic acid is L-ascorbic acid, D-ascorbic acid or DL-ascorbic acid, or salts thereof.

Accordingly, in another embodiment of the present invention, there is provided a composition for promoting osteoclastogenesis in vitro or in vivo, the composition comprising: a combination of an osteoclastogenic agent, a protein stabilizing agent and a cement, in amounts that are sufficient to promote osteoclastogenesis.

Typically, the osteoclastogenic agent is RANKL. The protein stabilizing agent is trehalose.

Accordingly, in yet another embodiment of the present invention, there is provided a composition for inducing differentiation or tissue repair in vitro or in vivo, the composition comprising: an anabolic compound for enhancing the bioactivity of an inductive protein.

Typically, the anabolic compound is a pyruvate. The inductive protein is RANKL. The pyruvate and the RANKL are in a matrix. The pyruvate and the RANKL are in solution. The pyruvate and the RANKL are in combination with ascorbic acid.

Accordingly, in yet another embodiment of the present invention, there is provided a method of promoting osteoclastogenesis in vitro or in vivo, the method comprising: differentiating progenitor cells into osteoclasts In contact with or localized near the composition, as described above.

Typically, the progenitor cells are osteoclast precursor cells.

Accordingly in yet another embodiment of the present invention, there is provided a method of producing a dehydrated matrix suitable for storage, the method comprising: a) adding an aqueous solution of RANKL to a material, the aqueous solution containing a protein stabilizing agent and b) dehydrating the mixture of step a) so as to produce a dehydrated matrix.

Typically, the method further comprising combining in solution an antioxidant and RANKL. The method further comprising combining in solution pyruvate salt or pyruvic acid with RANKL. The method further comprising combining in solution pyruvate salt or pyruvic acid with an antioxidant. The material is brushite cement. The dehydrated RANKL can induce differentiation of primary or cell line osteoclast precursors upon rehydration. The antioxidant is ascorbic acid. The protein stabilizing agent is trehalose.

According to another embodiment of the present invention, there is provided a method of treating bone trauma in a subject, the method, comprising: implanting a matrix, as described above, adjacent to a site of the trauma so as to enhance bone formation adjacent to or within the implant.

Typically, the bone trauma results from a bone degenerative disease. The bone trauma results from surgery. The bone degenerative disease is osteoporosis, osteoarthritis, rheumatoid arthritis, or periodontitis. The bone trauma is a bone fracture.

Accordingly, in another embodiment of the present invention, there is provided use of a matrix, as described above, to enhance bone formation.

Accordingly, in another embodiment of the present invention, there is provided use of a matrix coated onto or within a metallic implant, as described above, to enhance bone formation.

Accordingly, in another embodiment of the present invention, there is provided use of the matrix, as described above, as a bone graft for osteoclast resorption of the material

Accordingly, in another embodiment of the present invention, there is provided a stabilized RANKL composition suitable for storage, the composition comprising a dehydrated mixture of RANKL and a protein stabilizing agent.

Accordingly, in another embodiment of the present invention, there is provided a stabilized RANKL composition suitable for storage, the composition comprising a dehydrated mixture of RANKL and an antioxidant.

### BRIEF DESCRIPTION OF THE DRAWINGS

Further aspects and advantages of the present invention will become better understood with reference to the description in association with the following Figures, wherein:

FIG. 1a is a graph Illustrating percentage of TRAP positives cells normalized to positive control (RANKL+/cement−);

FIG. 1b, c are photographs comparing TRAP staining of RAW264.7 cells (b) and TRAP+ multinucleated osteoclasts formed from RAW264.7 cells in the presence of cement and addition of RANKL in the medium (c);

FIG. 1d is a photograph illustrating actin (red) and nuclei (blue) visualization inside osteoclastic cells formed on the surface of brushite cement from RAW264.7 cells cultured with RANKL;

FIG. 2 are photographs of cylinders of brushite cement before in vitro experiments;

FIG. 3a is a graph illustrating the ability of RANKL loaded (800 ng) cement cylinders (40 mg) to differentiate 10×10⁴ RAW264.7 cells into osteoclasts after six successive 7 or 5 day culture periods. (Culture medium (1 ml) changed daily; cement cylinders transferred to fresh undifferentiated monocyte cultures after each datum point shown in the figure) (n=3, mean+/−sd)

FIG. 3b is a graph illustrating the ability of RANKL loaded (800 ng) cement (19 mg) set within the pores of sintered titanium beads to differentiate 10×10⁴ RAW264.7 cells into osteoclasts after one 7 day and two five day successive cultures with the same matrices (Culture medium (1 ml) changed daily; cement cylinders transferred to fresh undifferentiated monocyte cultures after each datum point shown in the figure.)

FIG. 3c are a series of photographs of sintered titanium beads on one half of a cylindrical sample (8 mm diameter) before (top) and after (bottom) impregnation with brushite (arrows indicate the brushite);

FIG. 3d is a graph illustrating the percentage of TRAP positives cells compared with positive control formed after 5 days of culture in the presence of calcium cross linked alginate (1 ml 3 wt % aqueous solution) and RANKL (800 ng) incorporated into alginate. (n=3, mean+/−sd);

FIG. 4 is a series of photographs showing TRAP+ multinucleated osteoclasts formed from RAW264.7 cells in the presence of (a,b) of daily addition of 50 ng RANKL, (c,d) in the presence of brushite after 33 days of culture coated with 800 ng of RANKL and (e,f) in the presence of metallic implant with brushite inside his porosity coated with 800 ng of RANKL after 7 days of culture;

FIG. 5 is a graph illustrating the percentage of TRAP positives cells after 5 days of culture in presence of RANKL stored at 37° C. for 7, 14, 21 and 35 days prior to use in cell culture experiments. (n=4, mean+/−sd), when compared with control not stored prior to use;

FIG. 6a is a graph illustrating percentage of TRAP positive cells formed after 5 days of culture in the presence of brushite cement with two different ways of associating RANKL; mixing RANKL during cement setting and topical adsorption after setting;

FIG. 6b is a graph illustrating percentage of TRAP positive cells formed after 5 days of culture in the presence of hydroxyapatite cement with two different ways of associating RANKL;

FIG. 7 is a graph illustrating the Effect of ascorbic acid on the efficacy of RANKL in differentiating primary bone marrow cells. Number of TRAP positive multinucleated cells (Small<100 μm, Big>100 μm) formed after 5 and 7 days of culture of primary mouse bone marrow cells in the presence of RANKL (50 ng/ml) and ascorbic acid (50 ng/ml) in the culture medium. (n=3, mean+/−sd);

FIG. 8 is a graph illustrating the effect of ascorbic acid addition to the cell culture medium on the efficacy of RANKL in differentiating RAW264.7 monocyte cell line. Percentage of TRAP positive cells formed after 5 days of culture of osteoclast precursors in the presence of RANKL (50 ng/ml) and ascorbic acid (50 ng/ml) in the culture medium;

FIG. 9 is a series of photographs showing TRAP+ multinucleated osteoclasts formed after 5 days of culturing of RAW264.7 monocyte cell line with RANKL alone (a, b) or in combination with ascorbic acid (c, d);

FIGS. 10a-f are a series of photographs showing the characteristics of osteoclastogenesis in the presence of calcium phosphate cement and trehalose;

FIG. 10g is a graph illustrating the percentage of TRAP positive cells formed after 7 days of RAW 264.7 cell culture in the presence or absence brushite cement cylinder coated or not coated with RANKL (800 ng), after treatment or not with RANKL (50 ng/ml), after addition to the culture medium or not of trehalose (300 mM) and normalized to the positive control (cement−/RANKL+/Trehalose−). Data are mean±SEM, n=3 independent experiments. (c) indicates that RANKL was coated onto brushite cement cylinder;

FIG. 11a is a graph illustrating the evaluation of the stability of RANKL solution stored for 7, 14, 21 and 28 days at room temperature (RT), as determined by capacity for osteoclastogenesis; p FIG. 11b Illustrates the percentage of TRAP positive cells formed after 7 days in RAW 264.7 cell culture in the presence either of RANKL, loaded brushite (RaB) or RANKL and trehalose loaded brushite (RaTB) cement cylinder and normalized to the positive control (culture medium+fresh RANKL). These two formulations were stored at RT during either 30 minutes (sa) or 1 day (1 d). Data are mean±SD, n=3 replicates. Thus for RaTB, the brushite cement (40 mg) includes the following solution soaked onto it: 800 ng RANKL in 16 μL phosphate buffered saline to which 1.8 mg of trehalose was added;

FIG. 11c is a representation of the culture processes used to determine the stability the different formulations during 4 successive 7 days monocyte cell cultures. RaB and RaTB formulations were stored at RT during either 30 minutes (sa) or 1 day (1 d).

FIG. 11d illustrates the percentage TRAP positive cells normalized to the positive control (culture medium+fresh RANKL) and formed after 4 successive 7 days culture periods of RAW 264.7 cell in presence of (RaB)sa, (RaB)1 d, (RaTB)1 d, (RaTB)sa. Data are mean±SD, n=3 replicates. The differences were evaluated by analysis of variance (ANOVA) with Fisher's probability least significant difference (PLSD) post hoc test and considered to be significant at p<0.05. RaB: 40 mg brushite cement+800 ng RANKL in 16 μL phosphate buffered saline. RaTB: 40 mg brushite cement+800 ng RANKL+1.8 mg Trehalose in 16 μl phosphate buffered saline sa: left 30 minutes at room temperature prior to use. 1 d: left 1 day at room temperature prior to use;

FIG. 12a and b are graphs showing an evaluation of the stability of RANKL-trehalose-coated brushite cement (RaTB) under different storage conditions. RaTB formulation (as above) was stored either for 1 day (1 d), 3 weeks (3 w) or 5 weeks (5 w) at ambient conditions (a), ambient conditions with light excluded (FIG. 8bi), at 4 ° C. or −20° C. with light excluded (FIG. 12bii), room temperature light excluded in air (A) or nitrogen (N) (FIG. 12biii), compared with positive control after 7 days culture. Data are mean±SD, n=3 replicates;

FIG. 13 is a graph Illustrating the number of TRAP positives cells formed after 7 days of mouse bone marrow cell culture in the presence of RANKL-coated brushite cement (800 ng) (Culture medium (0.5 ml) changed every two days; addition of ascorbic acid (50 μg/ml) (AA) was required for this culture to form osteoclasts) (n=3, mean+/−sd);

FIG. 14 illustrates the effect of sodium pyruvate concentration and on osteoclastic potential of RANKL and in combination with ascorbic acid after 7 days culture with RAW.264.7 cell line; and

FIG. 15 is a graph illustrating the number of TRAP positives cells formed after 7 days of RAW 264.7 cell culture in the presence of cylinder of brushite cement loaded either with RANKL alone (800 ng) or RANKL (800 ng) and ascorbic acid (μg). (Culture medium (1 ml) changed every two days) (n=3, mean+/−sd).

### DETAILED DESCRIPTION OF THE INVENTION

### Definitions

Unless otherwise stated, the following terms apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus the phrase “consisting of” indicates that the listed elements are required or mandatory and that no other elements may be present.

As used herein, the term “pro-osteoclastogenic” or “osteoclastogenesis” is intended to mean induced formation of osteoclasts.

As used herein, the term “osteoclastogenic agent” is intended to mean an agent or agents, which when used either singly or in combination can induce the formation of osteoclasts (also described herein as an inductive protein). One example of such as agent is Receptor Activator for Nuclear Factor κ B Ligand (RANKL). RANKL may be used in combination with, ascorbic acid, sodium pyruvate, to Induce the formation of osteoclasts.

As used herein, the term “an anabolic compound” is intended to mean a compound that is capable of enhancing the metabolic activity of a cell. Example of such compounds include pyruvate salts (sodium pyruvate), pyruvic acid and the like.

As used herein the term “induce or enhance osteoclast formation” is intended to mean to cause osetoclast precursors a) to fuse forming multinucleated cells, b) to express specific proteins, such as tartrate-resistant acid phosphatase (TRAP), c) and following a) and b) to acquire the ability to become functional osteoclasts.

As used herein, the term “osteoconductive” is intended to mean promotion of bone apposition onto the surface of a graft or implant, thereby functioning as receptive scaffold.

As used herein, the term “bone apposition” is intended to mean the formation of new bone on the bone surface.

As used herein, the term “matrix” is intended to mean a biomaterial capable of a) storing osteoclastic agent and b) releasing it in active form and quantity to induce osteoclastogenesis.

As used herein, the term “matrix osteointegration” is intended to mean bone ingrowth into the porous surface of a matrix, or bone bonding to the surface of a matrix causing anchorage of the implant in the bone.

As used herein, the term “implantation site” is intended to mean a location in a subject's body where prosthesis, endoprosthesis, bone graft substitute, bone graft, soft tissue graft, are located to accelerate healing or to restore function to the musculoskeletal system including bone.

As used herein, the term “material” is intended to include natural and man-made materials, which are generally classed as metals, polymers, ceramics or composites thereof, and which are compatible for use in medical applications. The term “biomaterial” when used in conjunction with a matrix refers to a material that does not degrade the osteoclastogenic agent and is thus capable of releasing the agent in an active form in vitro or in vivo without adverse tissue or cell response.

As used herein the term “associated with” when referring to the relationship between the osteoclastogenic agent and the material, is intended to mean that the agent can be impregnated and/or coated onto and/or within the matrix. The agent is in an amount which when it is released from the matrix is in an amount which is sufficient to cause osteoclastogenesis.

As used herein, the term “metals” is intended to include biocompatible metals including, but not limited to, stainless steel, titanium, tantalum and nitinol.

As used herein, the term “polymer biomaterial” is intended to include two subclasses, namely polymers and hydrogels. Hydrogels are swollen polymer networks containing significant (>50%) quantities of water, (more typically>85%). Examples of hydrogels include crosslinked alginates, non-fibrillar collagens, PEG (polyethylene glycol), PAA (polyacrylic acid), HEMA (hydroxy ethyl methacrylate), and chitosan. Polymers include PE (polyethylene), PGA (polyglycolic acid), PLA (poly lactic acid), PU (polyurathanes), PHB (polyhydroxybutyrate), and PTFE (polytetrafluoroethylene), PVA (poly(vinyl alcohol)), cellulose.

As used herein, the term “ceramic” or “bioceramic” is intended to include hydroxyapatite, calcium phosphate, calcium hydrogen phosphate, calcium carbonate, calcium silicates, zeolites, artificial apatite, brushite, calcite, gypsum, phosphate calcium or, α and or β tricalcium phosphate, octocalcium phosphate, calcium pyrophosphate (anhydrous or hydrated), calcium polyphosphates (n≧3) dicalcium phosphate dihydrate or anhydrous, iron oxides, calcium carbonate, calcium sulphate, magnesium phosphate, calcium deficient apatites, amorphous calcium phosphates, crystalline or amorphous calcium carbonates, pyrophosphates and polyphosphates. Ceramics may contain one or more of titanium, zinc, aluminium, zirconium, magnesium, potassium, calcium, iron, and sodium ions or atoms in addition to one or more of an oxide; carbonates, carbides, nitrides, titanates, zirconates, phosphonates, sulphides, sulphates, selenides, selanates, phosphate, such as orthophosphate, pyrophosphate, di-phosphate, tri-phosphate, tetra-phosphate, penta-phosphate, meta-phosphate, poly-phosphate; a silicate.

As used herein, the terms “ceramic” or “bioceramic” are used interchangeably throughout and are intended to include all ceramics which may be formed from oxides, selenites, of calcium, sodium, potassium, aluminium, magnesium, zinc, silicon, strontium, barium, or transition metals. Bioceramics further include composites thereof with metallic, ceramic and polymeric phases that can be used for example as bone replacement. Any gel, such as a sol gel, xerogel, alcogel or aerogels and the like are also contemplated.

As used herein, the term “metallic implant” is intended to include an implant made from an elemental metal or alloys thereof, such as for example titanium and alloys nitinol thereof.

As used herein, the term “non-hydrogel polymer” is intended to mean polyurethane, polyester, polytetrafluoroethylene, polyethylene, polymethylmethacrylate, polysiloxanes, and all poly hydroxyacids. Examples of non-hydrogel polymers include, but are not limited to, the following synthetic and natural polymers:

| Synthetic polymers | Natural polymers |
|---|---|
| Poly lactic acid | Fibrin |
| Poly-L-lactic acid | Albumin |
| Poly-D,L-lactic acid | Casein |
| Poly glycolic acid | Keratin |
| Poly-e-caprolactone | Fibrillar Collagen |
| Poly-p-dioxanon | silk fibroin |
| Tri-methylen carbonate | lipids |
| Poly anhydrides | phospholipids |
| Poly ortho ester | Amphiphiles |
| Poly urethanes | Polyhydroxybutyric acid |
| Poly amino acids | |
| Poly hydroxy alcanoates | |
| Poly phosphazenes | |
| Polystyrenes e.g. Poly(styrene-co- | |
| chloromethylsytrene) | |
| lipids (e.g. monoolein) | |
| phospholipids | |
| Polyphosphoesters | |
| Polyphosphazenes | |
| Aliphatic Polyesters e.g. PCL PGA | |
| PLA & Copolymers | |
| PHB PHV & Copolymers | |
| Poly(1,4-butylene succinate), | |
| Nylons | |
| Non Hydrogel Polysaccharides, e.g. | |
| cellulose acetates | |
| PEG Based Polymers | |
| Poly(ethylene oxide) average | |
| Polyanhydrides | |
| Poly(butylene Terephthalate) | |
| Amphiphiles | |

As used herein, the term “treating bone trauma” is intended to mean treatment of a trauma associated with bone, as disclosed herein, in a subject, and includes the implantation of a matrix as described herein, adjacent to a site of the trauma so as to enhance bone formation adjacent to the implant to a subject.

As used herein, the term “subject” or “patient” is intended to mean humans and non-human mammals such as primates, cats, dogs, swine, cattle, sheep, goats, horses, rabbits, rats, mice and the like. In one example, the subject is a human.

I. Matrix and Composition

Our discovery concerns a matrix that is useful for inducing or enhancing osteoclast differentiation. The matrix comprises a material with an osteoclastogenic agent which is associated with the material. The osteoclastogenic agent is located in an amount which is sufficient to induce or enhance osteoclast differentiation.

Current clinical approaches for the treatment of osteoporosis are based on inhibition of osteoclast action, but this do not improve the quantity of bone. Without wishing to be bound by theory, we posit that localised induction of bone resorption by the release of pro-osteoclastogenesis molecules (osteoclastogenic agents) from the surface of the of the biomaterial or within the biomaterial may lead to a compensatory increase in bone formation, resulting in faster and better osteointegration of the matrix.

Generally speaking, we use matrices that are made of a set biomaterials. The biomaterial is typically microcrystalline and comprises crystals <500 μm in linear dimension. In order for the osteoclastogenic agent to be retained on and/or within the matrix. With the exception of hydrogels, the biomaterial generally has a porosity at greater than 5% relative porosity and a surface area of greater than 0.5 m² g⁻¹, which is sufficient for the osteoclastogenic agent to move out via the pores, by diffusion through the polymer network or dissolution of the biomaterial.

There are many examples of biomaterial which are contemplated for use as a matrix. In one example, the biomaterial is a ceramic or a non-metallic coating, such as a polymer coating carbon and the like. In one example used herein, the biomaterial is a calcium phosphate-based cement, however it is to be understood that the phosphate containing cement can be an orthophosphate, a pyrophosphate, a di-phosphate, a tri-phosphate, a tetra-phosphate, a penta-phosphate, a meta-phosphate, or a poly-phosphate, plaster, calcium silicate, calcium sulphate etc. In examples described herein, the cement sets to form mainly either brushite or hydroxyapatite.

The biomaterials comprise the osteoclastogenic agent, for example RANKL, which is impregnated onto and/or within the biomaterial.

The osteoclastogenic agent is combined with the biomaterial at a concentration of 1 ng or more and 1000000 ng or less of agent per mg of biomaterial. Typically, the osteoclastogenic agent is impregnated onto and/or within the biomaterial at a concentration of 10 ng or more and 500 ng or less of agent per mg of biomaterial. In one example, the osteoclastogenic agent is impregnated onto and/or within the biomaterial at a concentration of 15 ng or more and 25 ng or less of agent per mg of biomaterial. Cements are porous, which allows the RANKL to be absorbed into the cement

The combination may also be used with either brushite or hydroxyapatite as the biomaterial, although it is contemplated that other biomaterials as defined herein may also be used. In the examples described herein, the antioxidant is ascorbic acid. It is to be understood that ascorbic acid may be in either of its isomeric forms such as L-ascorbic acid, D-ascorbic acid or DL-ascorbic acid, or salts thereof. Furthermore, dehydroascorbic acid or salts thereof, can also be used.

The combination may also include pyruvates, such as sodium pyruvate and pyruvic acid, either singly or in combination with ascorbic acid. Without wishing to be bound by theory, we believe that the pyruvates provide energy to cells for cellular respiration and that compounds such as glucose, glucose phosphate and the like will also work.

Generally speaking, impregnated onto and/or within a material, the osteoclastogenic agent is in an amount that is sufficient to cause implant osteointegration, matrix remodelling, and osteoclast differentiation.

The aforesaid composition may be used to promote osteoclastogenesis in vitro or in vivo, in which the combination of RANKL ascorbic acid or sodium pyruvate are in amounts, which are sufficient to promote osteoclastogenesis in vitro or in vivo, when progenitor cells, generally osteoclast precursor cells, are contacted with the composition.

Ascorbic acid and sodium pyruvate are in the medium. It is contemplated that these compounds can be combined with the matrix with the RANKL

2. RANKL Stability

In vitro assays for osteoclast growth and resorption are useful for the screening of potential therapeutic agents for conditions such as osteoporosis. These assays rely on the use of RANKL that needs to be added, typically at least every 48 hours, to cultures in order for differentiation to occur and is typically stored at −80° C. prior to use. It is to be noted that RANKL's stability is rate determined. Thus it will degrade slowly over time. Typically, the stock is stored at −80° C. and solution are stored short term at −20° C. Our discovery is that a matrix comprising a single bioceramic pellet with RANKL adsorbed onto its surface causes osteoclastogenesis. However, RANKL is expensive and is unstable above −80° C.

In a clinical setting, degradable biomaterials are currently removed from the implantation site by virtue of being soluble in vivo or by being rendered soluble through hydrolysis or by enzymatic action. Bone autograft is a patient's own bone used as a graft. The graft is remodeled, that is, the graft recruits osteoclasts that erode the bone. The osteoclasts recruit osteoblasts that form new bone and gradually the graft is remodeled to become entirely new bone. By recruiting osteoclasts in vivo using our matrices coated with RANKL, may create a more autograft-like response.

To achieve this, we have demonstrated that RANKL, which is adsorbed in the pores of porous metals, is stable at ambient temperatures, specifically at 37° C. Specifically, the pores in porous metal are filled with calcium phosphate cement. RANKL is then soaked into that cement without a protein stabilizing agent.

This is achieved by drying the RANKL in the matrix. Furthermore the stability of RANKL in a dehydrated matrix could be enhanced using a sugar such as trehalose.

Thus, the aforesaid methods may also be adapted to include dehydrating an aqueous solution of RANKL and a protein stabilizing agent such as, for example, trehalose, so as to produce a dehydrated mixture of RANKL and the protein stabilizing agent. The trehalose is used to stabilise proteins during dehydration, thus the trehalose improves stability of RANKL loaded in brushite cement during storage. The ability of the dehydrated RANKL to induce differentiation of primary or cell line osteoclast precursors at any concentration can be measured using assays described herein.

3. Therapeutic Applications

Various models and studies have indicated that a short burst of osteoclastic activity is followed by a corresponding burst of osteoblast activity that results in a net gain in bone volume. Implants, which can induce the aforesaid osteoclast and osteoblast activity are likely to be highly valuable in accelerating implant fixation. Our matrix, in the form of an implant, may be useful to treat osteoporotic patients who often lack sufficient bone stock in which implants may be fixed.

As described below, we now believe that the initial stimulation of osteoclasts by RANKL leads to faster recruitment of more functionally active osteoblasts, resulting in better matrix integration and increase in bone mass. The combination of material and osteoclastogenic agent improves the recruitment of osteoclast at, adjacent to, or a distance away from the implantation site, and increases the quantity of surrounding bone. The combination might be very useful to treat numerous pathological diseases, which result in bone trauma, such as for example osteoporosis, osteoarthritis, rheumatoid arthritis, and periodontitis. The combination might also be useful to treat bone fractures, as well as to accelerate healing, or improve the quality of the bone formed, thereby increasing the success of surgeries, such as orthopaedic and maxillofacial surgery. Further uses include osteointegration, which is the induction of bone ingrowth into prosthesis stems, such as artificial joints and also artificial tooth roots. Bone formation, such as for example, bone grafting following non-union, treatment of osteoporotic fracture, trauma, or reconstruction after void filling following treatment of osteolysis, and spinal fusion, are also contemplated uses for the matrix. Thus, it is contemplated that the matrix may be used in a method of treating bone trauma in a subject, the method comprising: implanting the matrix adjacent to a site of the trauma so as to enhance bone formation adjacent to the implant. The bone trauma may result from a bone degenerative disease, such as osteoporosis, osteoarthritis, rheumatoid arthritis, or periodontitis, or the bone trauma may be a bone fracture or other injury.

4. In Vitro Applications

In vitro models of osteoclast formation are highly valuable for studying the cellular and molecular regulation of osteoclast differentiation and activation as well as pharmaceutical strategies to inhibit their action for treatment of osteoporosis. Principally these are cell lines based (often RAW 264.7) or primary cells treated with RANKL. A typical protocol involves the repeated addition of growth medium containing fresh 50 ng/ml soluble RANKL on the first, the third and the fifth day.

Our combination of RANKL with a biomaterial provides a new tool to simplify the current protocol by avoiding repeated addition of RANKL. Moreover, this material combined with RANKL is also of Interest to induce and study osteoclastogenesis during a long term.

### Materials and Methods

The following examples are offered by way of illustration, not by way of limitation. While specific examples have been provided, the above description is illustrative and not restrictive.

1. Preparation of Calcium Phosphate Cement Matrix.

1.1. Brushite Cement

Brushite cement powder was prepared from an equimolar amounts of calcium phosphate monohydrate (Mallinckrodt Baker, Germany) and β-TCP as described previously (Ionic modification of calcium phosphate cement viscosity. Part II: hypodermic injection and strength improvement of brushite cement: Barralet J E, Grover L M, Gbureck U. Biomaterials Volume: 25 Issue: 11 pages: 2197-2203; May 2004). The resulting powder was mixed with 0.8 M citric acid solution with a powder/liquid ratio 3.5 g/ml. The cement setting reaction is given by:

Ca₃(PO₄)₂+Ca(H₂PO₄)₂.H₂O+7H₂O→4CaHPO₄.2H₂O

For cell culture experiments brushite cement was set at room temperature in cylindrical molds to form 3 mm diameter and 3 mm height cylinders. The phase purity, the density and the specific surface area of brushite set cement were determined by X-ray diffraction by using a Siemens D5005 diffractometer (Siemens, Karlsruhe, Germany) with monochromated Cu Kα radiation, by using a helium pycnometer (AccPyc1330®, Micromeritics) and by using the Brunauer-Emmett-Teller (BET) method with helium adsorption-desorption (Tristar3000®, Micromeritics), respectively.

1.2 Alternative Cement Preparation

Either brushite or hydroxyapatite cements or brushite cement have been prepared. The brushite cement consisted of an equimolar mixture of β-TCP (tricalcium phosphate) and monocalcium phosphate hydrate. Powder and liquid was combined with a powder/liquid ratio 3.5 g/ml and was been moulded into cylinders 3 mm in diameter, 5 mm in length with an average weight of 40 mg. Hydroxyapatite cement consisted of an equimolar mixture of tetracalcium phosphate and dicalcium phosphate. Materials were washed 3 times in 70% alcohol and left under UV light for 12 hours. 16 μl (or 800 ng) of RANKL (50 μg/ml) was adsorbed onto these materials for 20 minutes.

1.3 Incorporation of RANKL Loaded Cement into Porous Metal

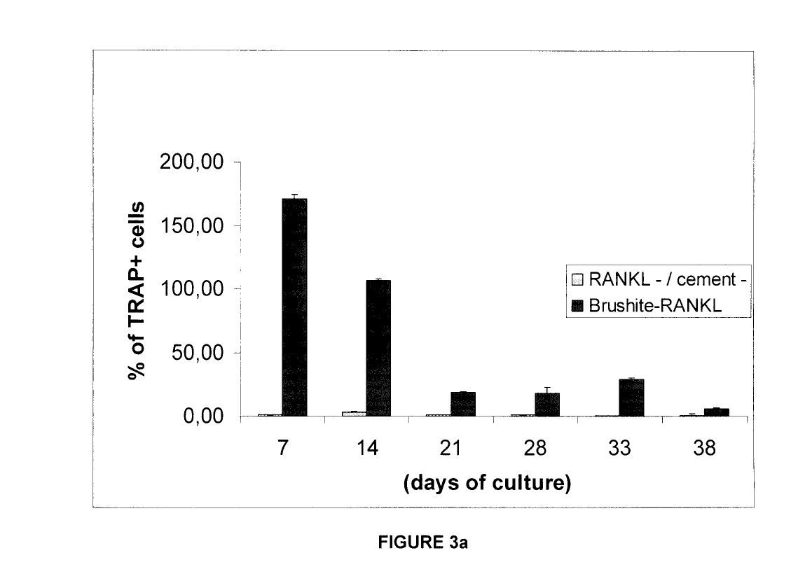

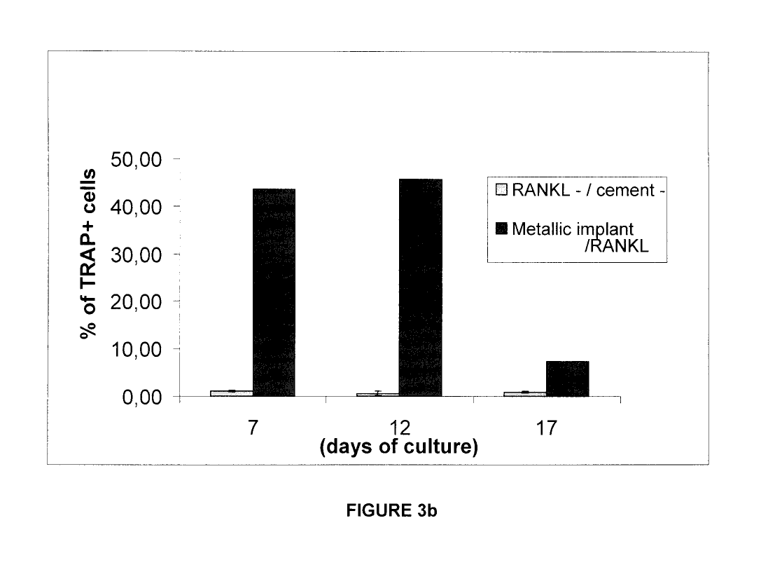

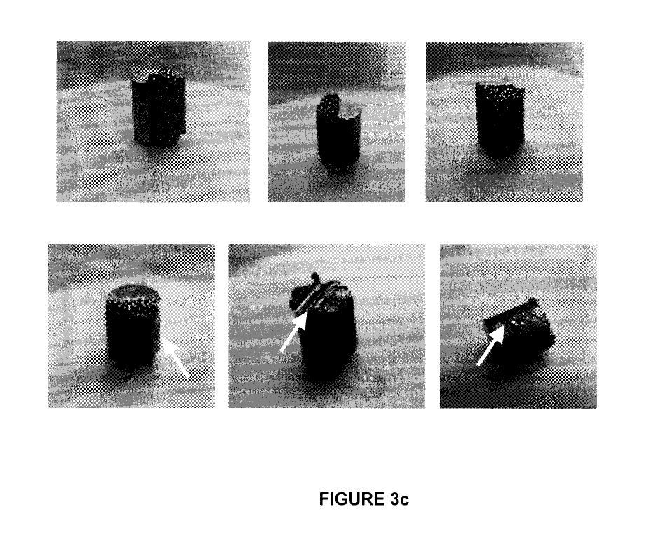

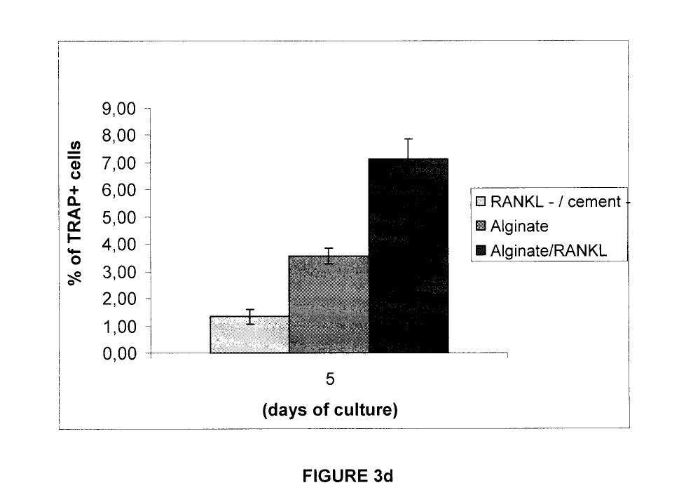

Porous titanium metallic implants 4 mm in diameter, 5 mm in length (FIG. 3c), had brushite cement paste infiltrated into the pores such that an average brushite weight of 11.2 mg was deposited in the pores. Once set, 800 ng of RANKL was soaked into the cement. 1.4 A 3 wt. % sodium alginate solution was prepared by mixing sodium alginate powder (MVG®, Pronova Biomedical a.s.) with double distilled water. The sodium alginate solution was sterilised by autoclaving (45 minutes at 121° C.) and cross-linked for 12 hours with 0.1 M CaCl₂ solution in the form of cylinders of 3 mm diameter and 3 mm height. Once the gels had ‘set’ 800 ng RANKL was injected into the gel using a hypodermic needle.

2a: Osteoclast Differentiation from Cell Line

RAW264.7 monocyte cell line were seeded at 2.5×10⁵ cells/cm², at day 0, and cultured for 5 to 7 days in 1 ml Dulbecco's Modified Eagle's Medium (DMEM) with 10% fetal bovine serum (FBS), 1% antibiotics and 1% sodium pyruvate at 37° C., 5% CO₂.

To induce osteoclast differentiation, either 1 μl of a pro-resorptive cytokine (RANKL (50 μg/mL)) was added to each ml of fresh medium every day following day 1, or materials combined with RANKL were added to cell culture at day 1. On day 5, cells were fixed using 4% paraformaldehyde during 10 minutes, washed 3 times with 1× PBS and stained for osteoclast marker TRAP, and the numbers of multinucleated, TRAP positive cells were assessed and cell number with matrices were compared with those in the positive control. Osteoclast resorption requires formation of specialized cytoskeletal structure, actin ring. To characterize actin organization in osteoclasts, we used BODIPY 581/591-conjugated phalloidin and DAPI to visualize F-actin and nuclei using fluorescence microscopy. Materials were used in subsequent experiments in the same conditions (without another addition of RANKL) to determine how long biologically active levels of RANKL can be release from these material. Stability of RANKL at 37° C. has also been tested.

2b: Osteoclast Differentiation from Bone Marrow

Mouse bone marrow cells were collected from mouse tibia and femora. Inbred mice (C57BL/6J, female, 6 weeks old) (Charles River Co., Wilmington, Mass., USA). Both femurs and tibia were dissected from dead mice under sterile conditions and immediately placed in sterile PBS solution. Under a laminar flow cabinet, the surrounding muscles were detached from the bones. Tibia and femurs were then cut in half and each part was placed in a different eppendorf tube and centrifuges (3 times, 5 seconds, 12,000 rpm). Bone pieces were removed and bone marrow was resuspended with 300 ml of medium (MEM) and collected. The bone marrow was then flushed with a 10 ml syringue with a 22-gauge needle in order to remove bone debris and blood clots. Nucleated cells were counted on Malassez haemocytometer slides. Cell viability was greater than 90% as determined by the trypan-blue dye exclusion test. The number of nucleated cells was around 80×10⁶ per ml of medium. Bone marrow cells were seeded at a final density of 10×10⁶ cells per cm² in 48 wells plate and cultured for 7 days at 37° C., 5% CO₂ with MEM supplemented with 10% FCS, 1% L-glutamine and 1% antibiotics. Medium was changed on day 1, 3 and 5 and 500 μl of fresh medium (MEM) added alone or supplemented with AA (50 μg/ml) and or sodium pyruvate (1-3%).

After 7 days of culture, mouse bone marrow cells were fixed with using 4% paraformaldehyde for 15 minutes, washed with phosphate buffered saline (PBS) and stained for osteoclast marker Tartrate-resistant acid phosphatase (TRAP). The cultured cells were stained for 10 to 20 minutes at 37° C. and the numbers of multinucleated TRAP positive cells were assessed using a light microscope (Eclipse TS100, Nikon, USA).

3. Incorporation of RANKL and RANKL-Trehalose Solutions onto Cement Matrix and Storage Conditions.

A recombinant glutathione S-transferase-soluble RANKL solution (50 μg/ml) and a D-(+)-trehalose dehydrate powder (Sigma-Aldrich, USA) were used in this study.

3.1. Stability of Receptor Activator for Nuclear Factor κ B Ligand (RANKL) Solution to Induce Osteoclast Formation.

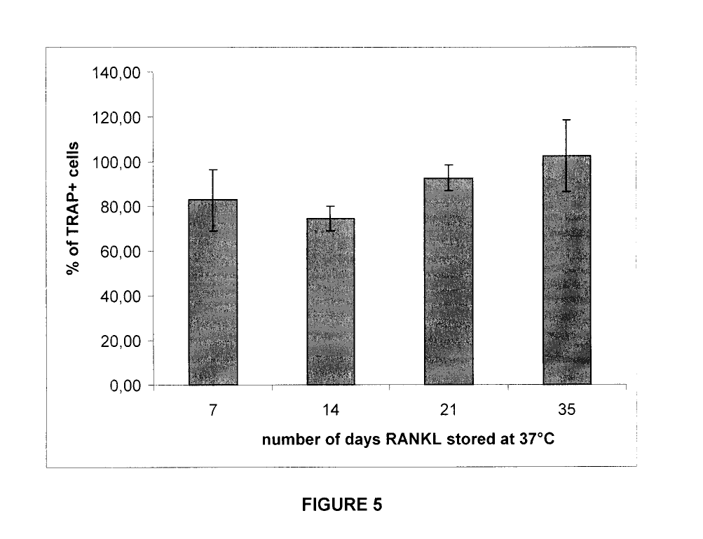

RANKL solution (50 μg/ml) was stored at room temperature (RT) for up to 5 weeks. After 1, 2, 3 and 5 weeks, osteoclast formation induced by stored RANKL solution was assessed and compared to osteoclast formation induced by a fresh RANKL solution (as seen in FIG. 5).

3.2. RANKL and RANKL-Trehalose Incorporation onto Materials.

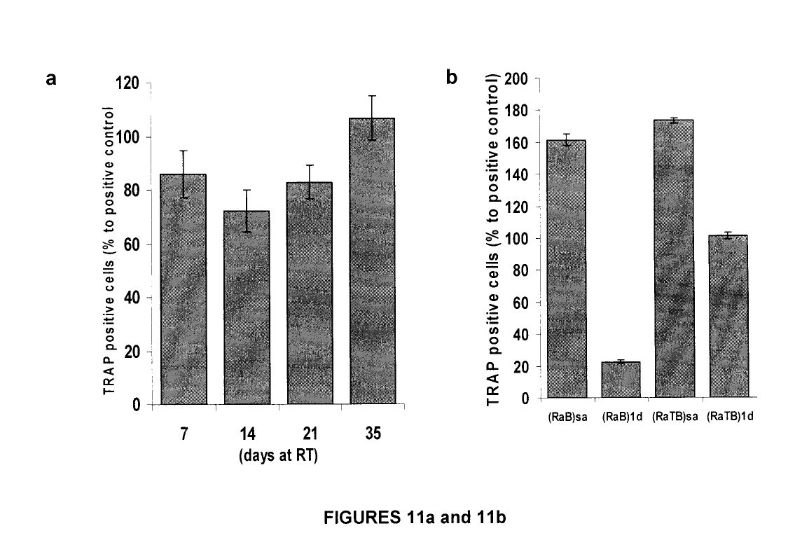

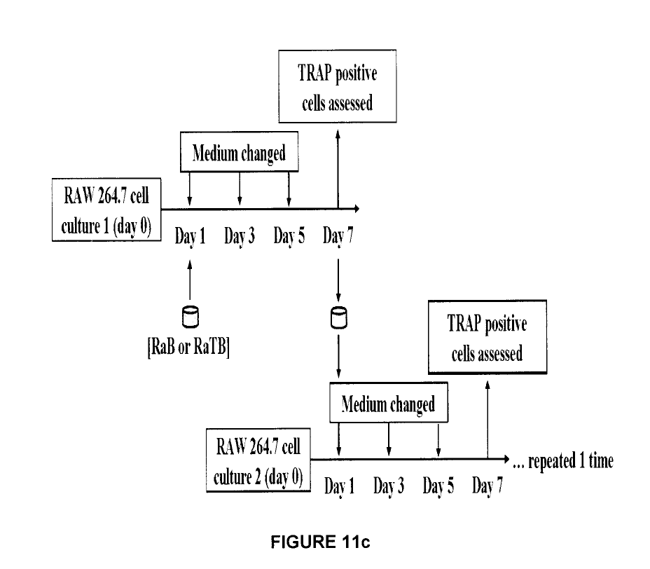

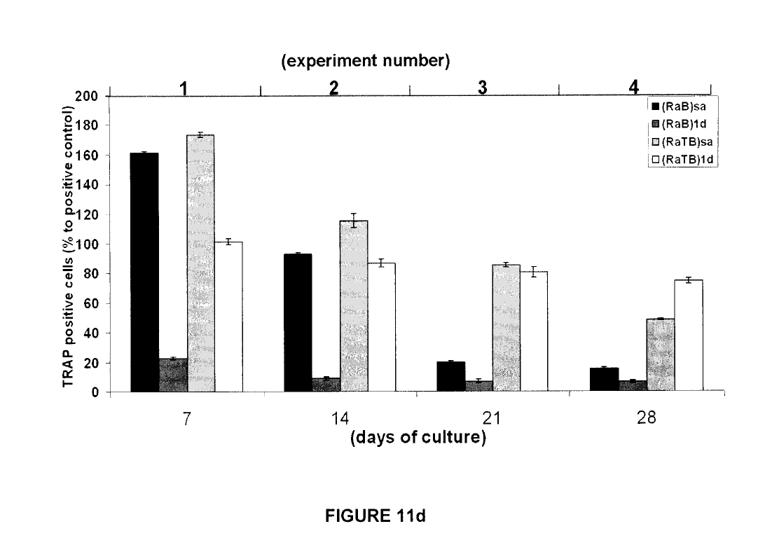

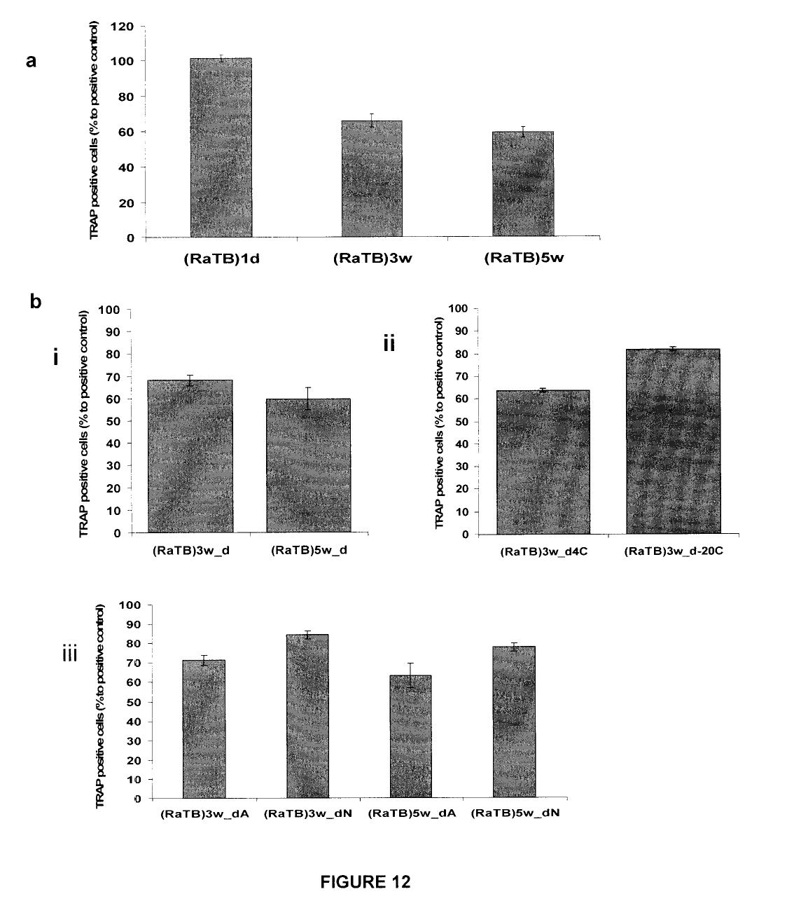

Brushite cement cylinders (B) (40 mg, 3 mm diameter and 3 mm height) were combined either with RANKL solution (50 μg/ml) alone (Ra) or with a RANKL-trehalose solution (RaT). The RaT solution was prepared by adding trehalose powder to 800 ng of RANKL solution to a final concentration of trehalose of 300 mM. Then Ra and RaT solutions were adsorbed onto the surface of set calcium phosphate cement. RaB and RaTB formulations were obtained and were stored at RT either for a short period of time of 30 minutes (sa) or over a long period time of 1 day, 3 weeks or 5 weeks, as seen in FIGS. 11 and 12)

3.3. Storage Conditions for the Long Adsorption Period.

To test the influence of different storage parameters on the stability of RANKL-trehalose solution loaded onto brushite cement cylinders, formulations were stored under specific conditions over a long period of time. Briefly, different times conditions (1 day (1 d), 3 weeks (3 w) or 5 weeks (5 w)), two different temperature conditions (4° C. (4) and −20° C. (−20 C)), two conditions of light exposure (protected from light (d) or not), and storage with dried air condition in the presence of silicate gel beads (A) or with pure nitrogen air condition (N) were compared, as seen in FIG. 12.

To assess the effects of trehalose addition and storage conditions on RANKL-Brushite, different formulations were prepared, added to the monocyte cell culture at day 1 and medium was changed at day 1, 3 and 5. In control cultures, osteoclastogenesis was induced addition of soluble RANKL (50 ng/ml) to fresh medium at day 1, 3 and 5. RAW 264.7 cells cultured without RANKL or biomaterial addition were used as the negative control.

On day 7, cells were fixed using 4% paraformaldehyde for 15 minutes, washed with phosphate buffered saline (PBS) and stained for osteoclast marker Tartrate-resistant acid phosphatase (TRAP). TRAP staining solution (4% solution of 2.5. M acetate buffer (pH=5.2), 12.5 mg/ml naphthol AS-BI phosphoric acid, 0.67 M tartrate buffer (pH=5.2), and 15 mg of fast Garnet salt) was freshly prepared and filtered before use. The cultured cells were stained for 10 to 20 minutes at 37° C. and the numbers of multinucleated TRAP positive cells were assessed using a light microscope (Eclipse TS100, Nikon, USA).

3.4. Time Stability of RANKL and RANKL-Trehalose-Coated Brushite Cement

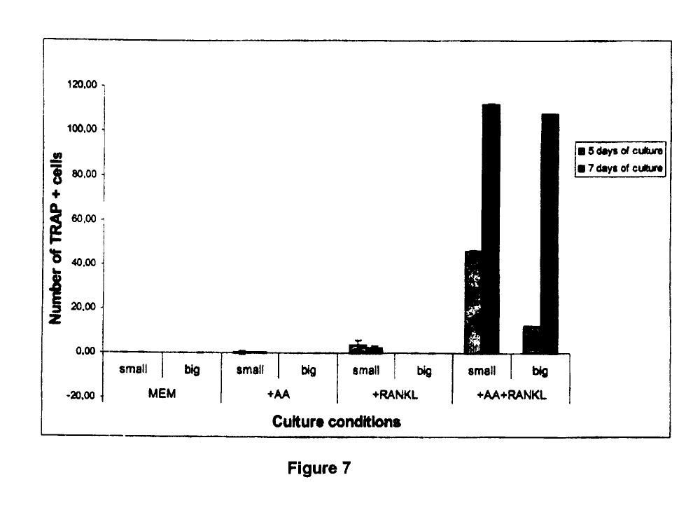

Cylinders of brushite cement coated either with 800 ng of RANKL only (RaB) or with 800 ng of RANKL mixed with 300 mM trehalose (RaTB) were used during four consecutive 7 days RAW264.7 cell cultures for a total of 28 days. Before using these two formulations both RaB and RaTB were stored either for a short period of time of 30 minutes (se) or for a period of time of 1 day (1 d). RAW 264.7 cells at a density of 2.5×10⁵ cells/cm² with 1 ml of medium were cultured. The four different formulations, (RaB)sa, (RaB)1 d, (RaTB)sa and (RaTB)1 d were added to the cell culture at day 1. Cells were cultured during 7 days at 37° C. and medium was changed at day 1, 3 and 5. At the end of this culture period, we transferred the cylinders of brushite cement to freshly plated monocyte cell cultures and we assessed the number of TRAP positive cells. This process was repeated four times. The results are shown in FIG. 7d.

4. Statistical Analysis.

All data were expressed as mean±standard deviation or standard error of the mean. The differences were evaluated by analysis of variance (ANOVA) with Fisher's probability least significant difference (PLSD) post hoc test and considered to be significant at p<0.05.

Results

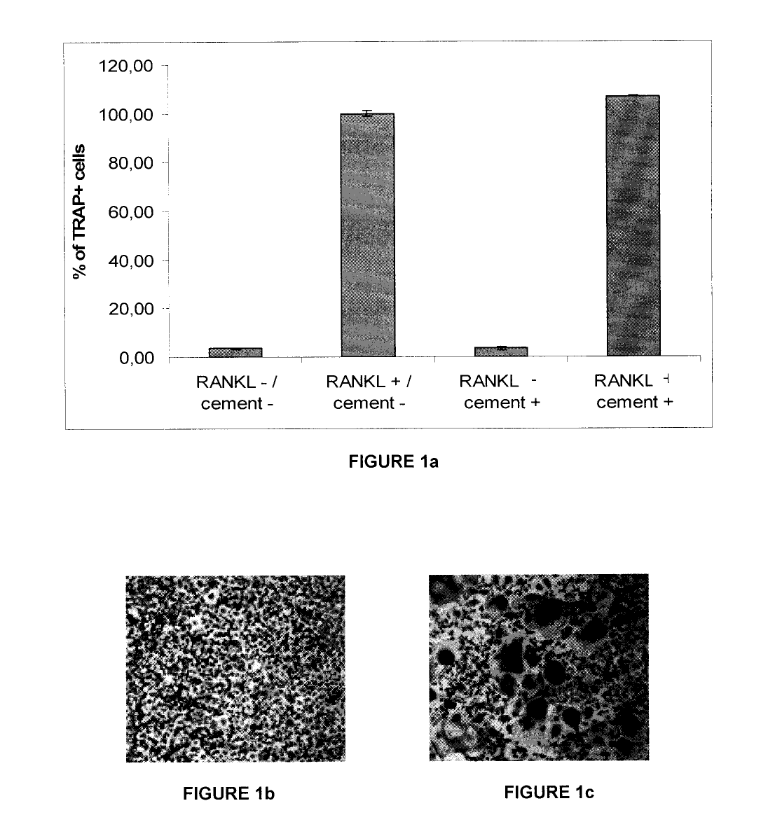

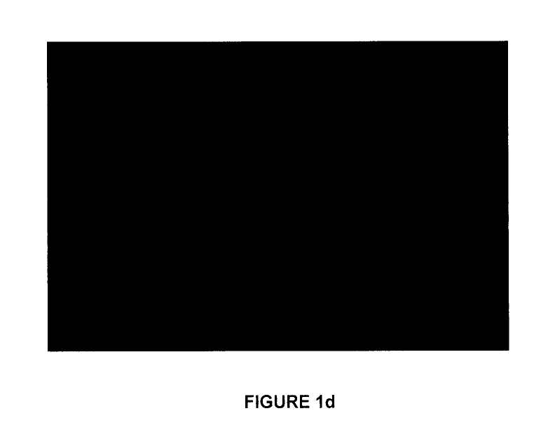

As illustrated in FIG. 1a, there is differentiation of RAW264.7 cells toward osteoclast-like cells on cement with addition of RANKL in the medium. Since the brushite cement is opaque, we assessed the numbers of osteoclasts formed on the plastic surrounding and underlying the cement and compared to the numbers of osteoclasts formed in the absence of the cement. RANKL induced osteoclast formation to a similar extent was independent of the presence of cement in the well. Osteoclast differentiation in the presence or absence of brushite cement was observed only in the presence of RANKL as illustrated in FIGS. 1b and 1c. These data Indicate that the brushite cement exhibits neither a detrimental nor stimulatory effects of on osteoclast formation.

As illustrated in FIG. 1d, fluorescently labeled osteoclasts are formed directly on the cement. In the presence of RANKL, osteoclasts formed exhibited an actin ring surrounding numerous nuclei. Without RANKL, no actin ring formation was observed. These data indicate that cement supports adhesion and formation of functional osteoclasts.

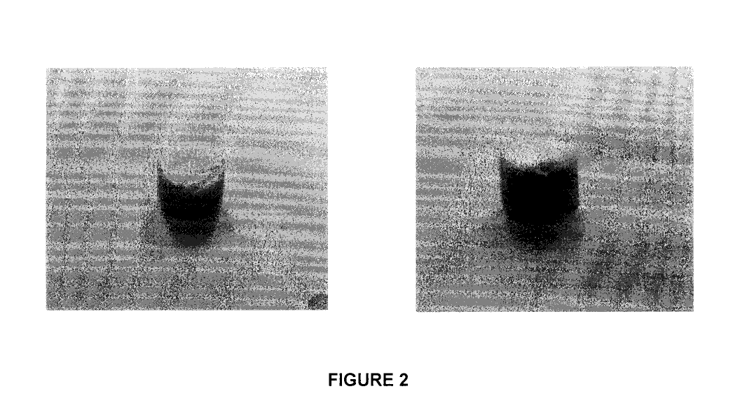

Table 1 below shows different tests performed by coating increasing amount of RANKL onto small (40 mg) cylinders of brushite as illustrated in FIG. 2. After 7 days of culture in presence of RANKL coated brushite cylinders, the number of wells in which RAW264.7 were differentiated toward osteoclastic-like cells, were noted as “positive”. As culture in the presence of RANKL in the medium, brushite material coated with at least 600 ng of RANKL induced differentiation of RAW264.7 cells. No significant difference was observed between materials coated with 800 ng or 1000 ng of RANKL. The following experiments were realized with an amount of RANKL of 800 ng to obtain the optimal response.

TABLE 1
Effect brushite materials coated with increasing coating amount of RANKL
onto RAW264.7 cells differentiation.

| | Amount of RANKL coated (ng) | | | | | | | | | |
|---|---|---|---|---|---|---|---|---|---|---|
| | 50 | 100 | 150 | 200 | 250 | 300 | 450 | 600 | 800 | 1000 |
| number | 0/2 | 0/2 | 0/2 | 0/2 | 0/2 | 0/2 | 0/2 | 1/2 | 2/2 | 2/2 |
| of positive wells | | | | | | | | | | |

Thus the matrix comprises the osteoclastogenic agent which is adsorbed onto the surface of the biomaterial at a concentration of 1 ng or more and 1000000 ng or less of agent per mg of biomaterial. In one example, the agent is at a concentration of 10 ng or more and 500 ng or less of agent per mg of biomaterial. In another example, the agent is at a concentration of 15 ng or more and 25 ng or less of agent per mg of biomaterial.

FIG. 3a shows the number of osteoclastic-like cells found after 7, 14, 21, 28, 33 and 38 days of culture of RAW264.7 cells in presence of brushite cement cylinder impregnated with 800 ng of RANKL. FIG. 3b shows the number of osteoclastic-like cells, which were found after 7, 12 and 17 days of culture of RAW264.7 cells in presence of a macroporous titanium sample with brushite cement loaded within the macroporosity. 19.2 mg amounts of brushite were incorporated inside the porosity of the metallic material. RANKL (800 ng) was coated onto the metallic/brushite material as shown in FIG. 3c. FIG. 3d shows the number of osteoclastic-like cells found after 5 days of culture of RAW264.7 cells in presence of calcium cross linked sodium alginate and alginate with RANKL incorporated within it (800 ng).

For all these conditions and materials, the numbers of osteoclasts formed on the plastic surrounding and underlying brushite based materials were assessed.

These data indicate that the brushite based materials continuously release amounts of RANKL in the medium to induce differentiation of RAW264.7 cell toward osteoclastic-like cells. Even after 38 days of culture, the materials still have the capacity to release enough RANKL to induce formation of osteoclast-like cells. Nevertheless, Alginate, a hydrogel polysaccharide material, with the same amount of RANKL incorporated within it, did not induce a significant differentiation of RAW264.7 toward osteoclastic cells.

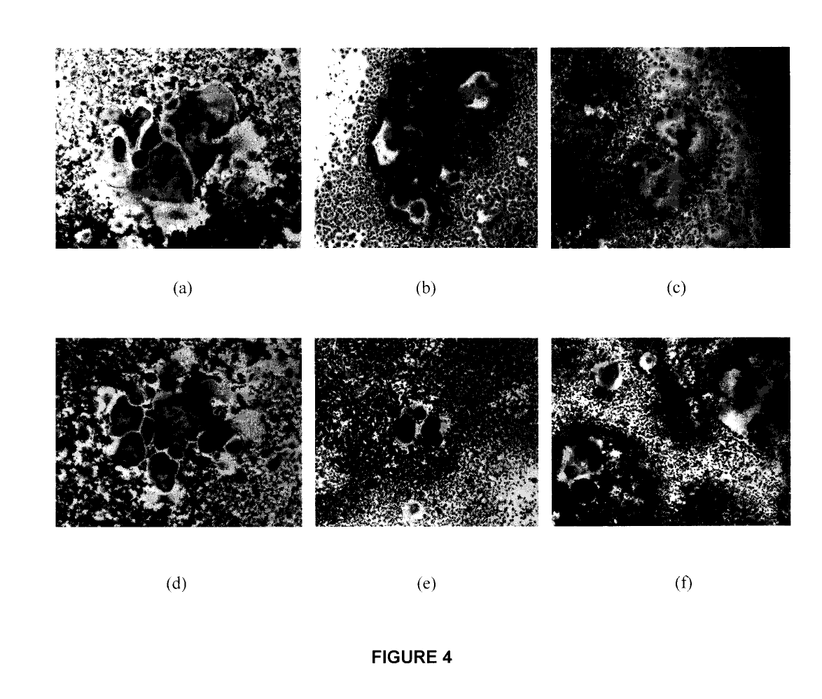

Osteoclast differentiation was observed in all conditions, with brushite cement cylinder coated with 800 ng of RANKL (see FIGS. 4c and 4d) and with the metallic/brushite material coated with 800 ng of RANKL (see FIGS. 4e and 4f). Osteoclast formation was compared with osteoclast formation observed after culture in a fresh medium daily supplemented with 50 ng/ml of RANKL (see FIGS. 4a and 4b).

TRAP-positive multinucleated osteoclasts formed from RAW264.7 cells in the presence of daily addition of 50 ng/ml RANKL (as illustrated in FIGS. 4a and 4b), in the presence of brushite after 33 days of culture coated with 800 ng of RANKL (as illustrated in FIGS. 4c and 4d) and in the presence of a macroporous metallic implant with RANKL loaded brushite inside the macropores (800 ng of RANKL) after 7 days of culture (as illustrated in FIGS. 4e and 4f).

As illustrated in FIG. 5, RANKL stock solution (50 μg/ml in 1× PBS) is stable after 7, 14, 21 and 35 days of storage at 37° C. compared to fresh RANKL solution. These data indicate that even after 35 days of storage at 37° C., RANKL is still an active molecule and able to induce RAW264.7 cell differentiation toward osteoclast-like cells. These data also suggest that RANKL coated onto and released from our materials is stable for a long time at ambient temperature and could induce osteoclastic differentiation in a long succession of in vitro experiments.

Overall data suggest that microcrystalline bioceramics such as brushite or apatite based materials are suitable as a matrix for the release of RANKL and induce differentiation of RAW264.7 cells toward osteoclastic cells. This release is continuous with time and the amount of RANKL released is in amounts which are sufficient to induce osteoclast formation, even after 33 days of culture.

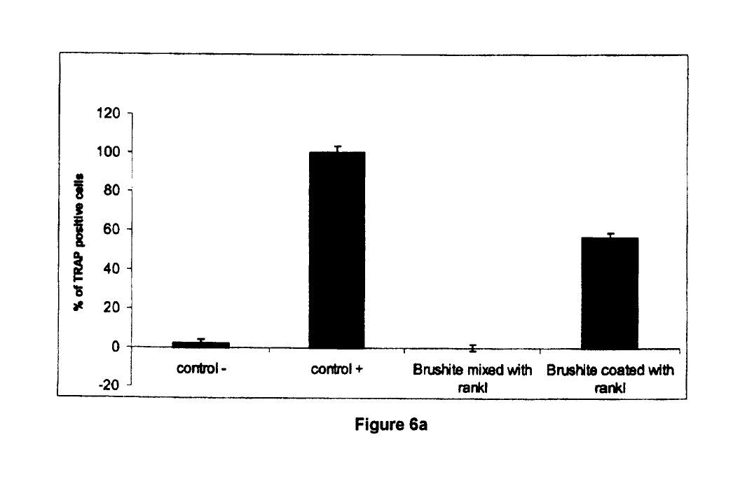

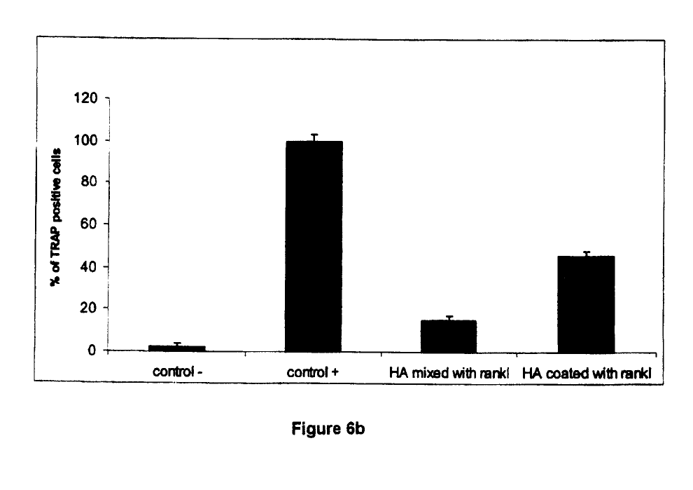

Brushite cement is composed of powder (tricalcium phosphate) and a liquid component (phosphoric acid solution and a retardant). After mixing, a paste is obtained and set in about 10 minutes. Besides incorporating the RANKL solution within the set brushite cement, another way of incorporating RANKL into brushite cement was to mix RANKL (800 ng) to the liquid component (20 μL 3 M H₃PO₄+500 mM citric acid) and mix this solution with 40 mg powder to obtain a setting cement. These two ways of incorporation of RANKL (namely by mixing during the preparation of the cement or by coating after the setting) were tested. No TRAP positive cell was observed for the culture when brushite cement made with a liquid component containing RANKL was added to cultures. TRAP positive cells were only observed when preset brushite cement was coated with RANKL (see FIG. 6a).

Hydroxyapatite cement is composed of powder (e.g. equimolar mixture of tetracalcium phosphate and dicalcium phosphate anhydrous) and a liquid component (water and an accelerator). After mixing, a paste is obtained and set in about 10 minutes. Besides incorporating the RANKL solution within the set hydroxapatite cement, another way of incorporating RANKL into hydroxyapatite cement was to mix RANKL (800 ng) to the liquid component (18 μL sodium phosphate solution) and mix this solution with 40 mg powder to obtain a setting cement. As illustrated in FIG. 6b, these two ways of incorporation of RANKL (namely by mixing during the preparation of the cement or by coating after the setting) were tested. One third of the percentage of TRAP positive cells was observed when hydroxyapatite cement made with a liquid component containing RANKL compared with set hydroxyapatite cement which was coated with RANKL. Moreover, much less differentiation was observed compared with a culture to which 50 ng of RANKL solution was added to the cell culture medium (positive control)

Any microporous material or material capable of storing and releasing protein without significant loss of protein biological activity Is likely to work similarly provided the specific surface area is adequate RANKL adsorption and/or the relative porosity is high enough that the pores may act as storage depots. Typically, crystals would have to be less than 1000 μm in linear dimension for an adequate specific surface area and relative porosity would need to be greater than 5% ion order for sufficient open porosity to exist in the material. The porosity is at >5% relative porosity, which is relative to full density (i.e. zero porosity).

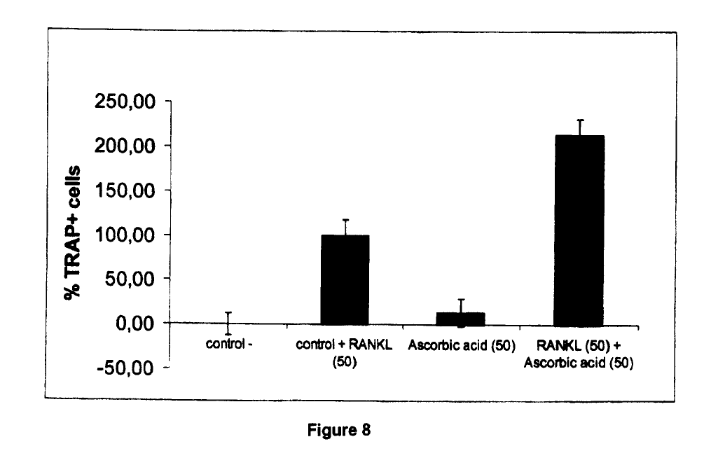

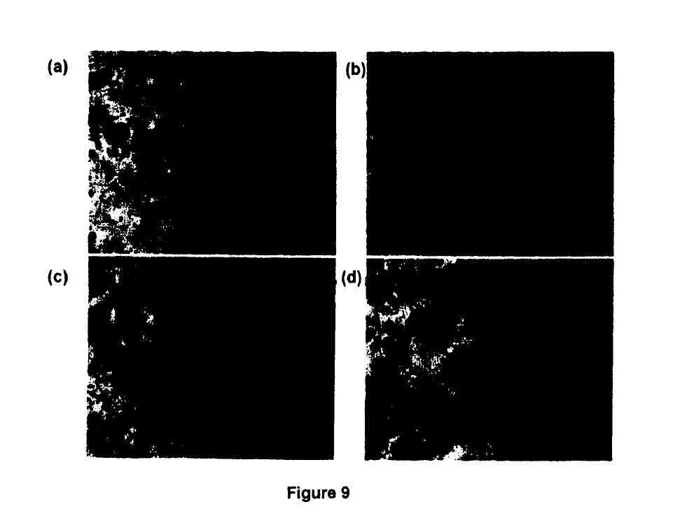

In addition to RANKL (1 μl of a 50 μg/ml RANKL stock solution) in fresh culture medium (1 ml), ascorbic acid (1 μl of a 50 μg/ml ascorbic acid stock solution) improves osteoclastogenesis from both primary mouse bone marrow cells (FIG. 7) and RAW264.7 cells (FIG. 8). This combination decreased the time to form osteoclasts and increased both the number and the size of osteoclasts (FIG. 9).

The ascorbic acid used may be L-ascorbic acid, D-ascorbic acid or DL-ascorbic acid, or salts thereof. Additionally, dehydroascorbic acid or salts thereof is also contemplated. Examples of other antioxidants are also contemplated such as, but not limited to, thiols, phenols, glutathione, vitamin E, catalase, super oxide dismutase, peroxidases and cofactors thereof, lipoic acid, uric acid, carotenes, lipid β-carotene, retinol, or ubiquinone.

Data indicate that when RANKL and ascorbic acid were coated onto two different brushite cement cylinders, differentiation of RAW264.7 cell toward osteoclastic-like cells after 5 to 7 days of culture. RANKL (800 ng) and RANKL (800 ng)/ascorbic acid (800 ng) loaded brushite cement were tested to reproduce previous data. Addition of both RANKL coated brushite cement and ascorbic acid coated brushite cement induced much greater formation of osteoclasts after 5 to 7 days of culture than the RANKL coated brushite cement alone (see FIG. 11).

Long term conservation at room temperature (24 hours at least) of RANKL: (800 ng) or RANKL (800 ng) loaded cement (40 mg) was tested by addition or not of trehalose (300 mM) to the pro-osteoclastogenic molecules before coating. Incorporation of RANKL with or without addition of trehalose was tested. TRAP positive cells were observed in all RANKL culture conditions with addition of trehalose.

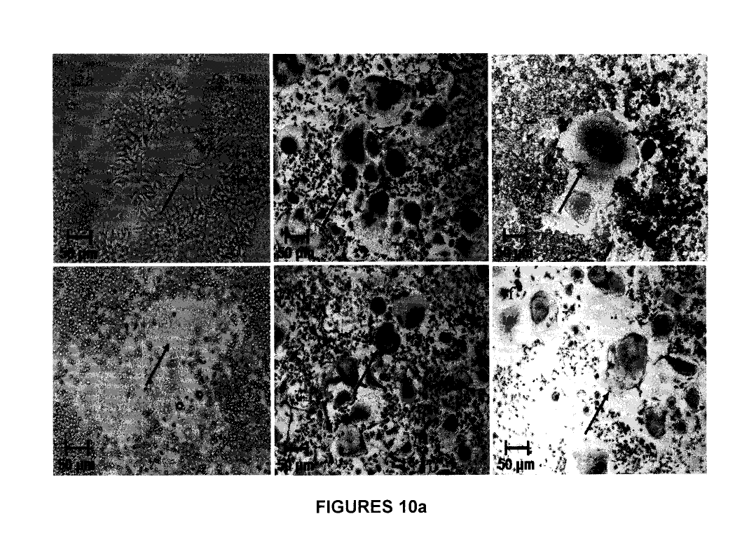

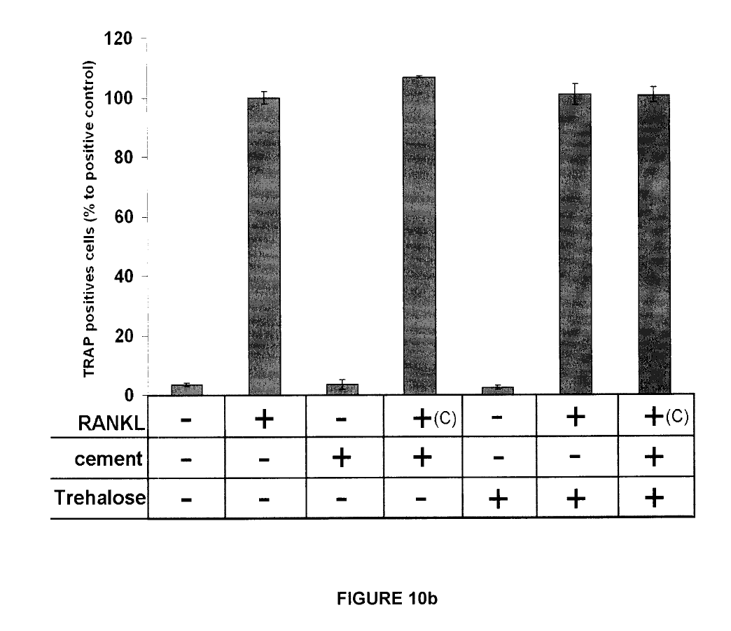

The effects of brushite cement and trehalose on osteoclast formation from monocyte cell culture were observed. The morphology and the number of osteoclast formed after 7 days of RAW 264.7 cell culture in the presence of loaded or unloaded material with RANKL and in the presence or not of trehalose were analyzed. Monocyte cell culture treated with soluble RANKL (50 ng/ml) or in the presence of RANKL-coated brushite cement formed multinucleated TRAP positive osteoclastic cells at the same level (FIGS. 10a, b and c). Addition of trehalose (300 mM) to the different culture conditions did not affect either positively or negatively the osteoclastogenic process induced by soluble RANKL or RANKL-coated brushite material (see FIGS. 10d to g)

Stability of RANKL solution alone or coated onto brushite cement and effects of trehalose addition to the coated RANKL solution were evaluated. We first studied the stability of RANKL to induce osteoclast formation (FIG. 11a). After 7, 14, 21 and 35 days of storage at RT, RANKL solution induced a percentage of osteoclastogenesis of 85.90±8.65%, 72.12±7.98%, 82.93±6.29% and 106.71±8.26% compared to the positive control (fresh RANKL solution) respectively. Thus, RANKL solution retained a high osteoclastogenic activity comparable to the positive control even after 35 days of storage at RT.

We next investigated the stability of RANKL (800 ng) coated onto the surface of cement cylinders and the effect of trehalose addition (300 mM) to the coated RANKL formulation. RANKL-Brushlte cement (RaB) and RANKL-trehalose-Brushite cement (RaTB) were stored during 30 minutes (sa) or 1 day (1 d) prior to use in monocyte cell culture (FIG. 11b). After 7 days of culture, (RaB)sa induced in this study a percentage of osteoclast formation of 161.50±3.79% of positive control. In contrast, the same formulation stored during 1 day induced a percentage of osteoclast formation significantly lower with 22.54±1.00% of positive control. Addition of trehalose to RaB formulation stored either during 30 minutes ((RaTB)sa) or during 1 day ((RaTB)1 d) significantly increased the osteoclastogenic processes to 173.48±1.53% and 101.41±2.00% of positive control respectively

Then we determined how long trehalose could preserve the bioactivity of RANKL in (RaB)sa, (RaB)1 d, (RaTB)sa and (RaTB)1 d formulations. We compared the stability of these four formulations to induce osteoclast formation during four consecutive monocyte cell cultures (FIGS. 11c and d). The percentage of TRAP positive cells induced by (RaB)sa formulation continuously decreased from 161.50±3.79% after the first 7 days culture period to 15.18±2.08% of positive control after 28 cumulative days of culture. After the first culture period, (RaB)1 d formulation induced an osteoclast significantly lower with 22.54±1.00% of positive control. During the next three cell cultures, the percentage of TRAP positive cells decreased from 8.81±1.15% after 14 days to 6.50±1.00% of the positive control at the end of the fourth culture period. Addition of trehalose to (RaB)sa formulation significantly increased the osteoclastic formation induced at the end of each culture period compared to (RaB)sa with percentages of TRAP positive cells from 173.48±1.53% to 48.48±0.58% of positive control. In contrast addition of trehalose did not change the decreasing trend of osteoclastogenic activity observed during these four culture periods with (RaB)sa. In the same manner the osteoclast formation induced at the end of each culture period by (RaB)1 d was significantly increased by the addition of trehalose to the formulation with percentages from 101.41±2.00% at the end of the first culture period to 74.80±2.00% after 28 cumulative days of culture. Interestingly, after the third culture period, the percentage of TRAP positive cells induce by (RaTB)1 d and (RaTB)sa were non significantly different. Moreover, at the end of last culture period, the percentage of TRAP positive cells induce by (RaTB)1 d was significantly higher than the percentage of TRAP positive cells induced by (RaTB)sa. Thus, RANKL-Brushite cement cylinder retained a higher osteoclastogenic activity during 4 weeks with addition of trehalose to (RaB) formulations.

We investigated the effects of different parameters of storage on the osteoclastogenic activity of RANKL-trehalose-loaded brushite cement. First the stability RANKL-trehalose-coated brushite cement formulation (RaTB) stored during increased time period (1 day(1 d), 3 weeks (3 w) and 5 weeks (5 w)) to induce osteoclast formation was assessed (FIG. 12a). After 7 days of monocyte cell culture, (RaTB)1 d formulation induced an osteoclastogenic process comparable to the positive control. The percentage of osteoclast formation induced by (RaTB) stored during 3 weeks (RaTB)3 w and 5 weeks (RaTB)5 w significantly decreased compared to (RaTB)1 d with 65.92±3.77% and 59.35±2.87% of positive control respectively. After 3 weeks and 5 weeks of storage RaTB formulations retained significantly the same osteoclastogenic activities. There was no further deterioration of the osteoclastogenesis after 3 weeks.

To determine the parameters responsible of the decrease of efficiency of RaTB formulation to induce osteoclast differentiation, we first tested the effect of light exposure during the storage. We assessed the osteoclastogenic activity of both (RaTB)3 w and (RaTB)5 w formulations stored protected from light (d) after 7 days of RAW 264.7 cell culture (FIG. 12bi). The percentage of osteoclast formation induced by (RaTB)3 w_d was higher but not significantly different form the percentage induced by (RaTB)5 w_d formulations with 68.18±2.38% and 59.74±4.97% of positive control respectively. Moreover, the osteoclastogenic activities of (RaTB)3 w and (RaTB)5 w formulations stored protected from light or not were not significantly different (FIG. 12a). Thus, light exposure during the storage did not significantly influence the osteoclastogenic stability of the RaTB formulation.

Then we investigated the effect of temperature on the stability of RaTB formulation to induce osteoclast formation. (RaTB) formulation was stored protected from light during 3 weeks either at 4° C. ((RaTB)3 w_d4C) or at −20° C. ((RaTB)3 w_d 20C) (FIG. 12bii). After 7 days of monocyte cell culture, the percentage of TRAP positive cells induced by (RaTB)3 w formulation stored at 4° C. was 63.64±0.82% of positive control. The percentage of TRAP positive cells induced by the same formulation stored at −20° C. (81.82±0.82% of positive control) was significantly higher from the percentage of TRAP positive cells formed both by (RaTB)3 w_d4C formulation and by (RaTB)3 w formulation (FIG. 12a). Thus, the stability of (RaTB)3 w formulation to induce osteoclast formation was increased by a storage temperature condition of −20° C.

Next, we tested the effects of atmosphere composition on the stability of our formulation during the storage (FIG. 12biii). (RaTB was stored protected from light during 3 weeks and 5 weeks either in the presence of dried (with silica gel) air (A) or in the presence of nitrogen gas only (N). After 3 weeks of storage, RaTB formulation induced an osteoclast formation significantly higher in the presence of nitrogen gas only than in the presence of dried air with percentages of osteoclast formation of 84.27±2.08% and 71.28±2.52% of positive respectively. The same trend was observed with RaTB formulation stored during 5 weeks and after 7 days of monocyte cell culture (RaTB)5 w_dN induced a percentage of TRAP positive cells significantly higher than the percentage of TRAP positive cells formed by (RaTB)5 w_dA with 77.63±2.08% and 63.20±6.24% of positive control respectively. The osteoclastogenic process induced by (RaTB)3 w_dN was significantly higher than the osteoclastogenic process induced by (RaTB)5 w_dN but percentages of osteoclast formation induced both by (RaTB)3 w_dN and by (RaTB)5 w_dN were significantly higher than the percentages of osteoclast formation induced by (RaTB)3 w and (RaTB)5 w formulations respectively (FIG. 12a). Thus, absence of oxygen during the storage of RANKL-trehalose-brushite formulation for 3 weeks and 5 weeks significantly increased the osteoclastogenic activity of these formulations.

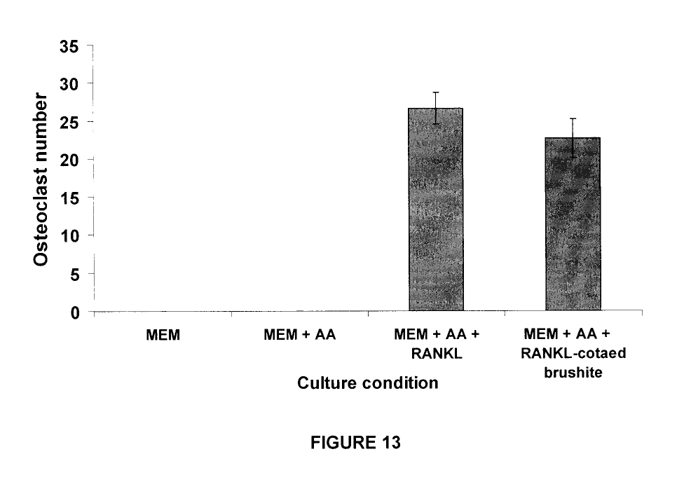

We tested the ability of brushite cement cylinder loaded with RANKL to induce osteoclast formation from a primary culture of mouse bone marrow cells. Mouse bone mouse cells with RANKL in solution or combined with a cement matrix for 7 days and formation of osteoclastic cells was only observed either in the presence of addition of RANKL and ascorbic acid in the medium or in the presence of RANKL coated brushite cement and addition of fresh ascorbic acid to the medium (FIG. 13). For these two conditions, the number of TRAP positive cells was not statically different.

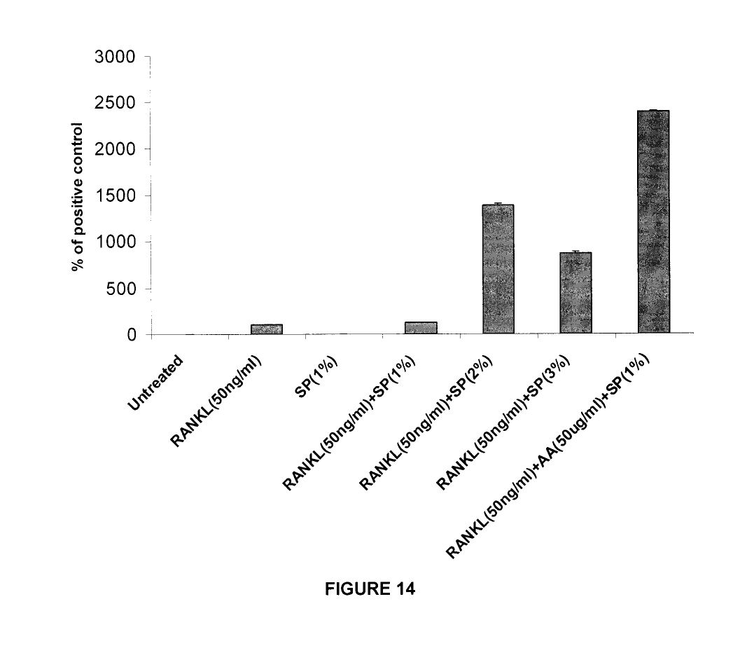

We investigated the effect of sodium pyruvate on RANKL osteoclastogenesis from monocyte cell line with and without ascorbic acid. RAW 24.7 cells were culture for 7 days in different conditions in the presence of RANKL, ascorbic acid, sodium pyruvate and various combinations of these factors (FIG. 14). Addition of sodium pyruvate (1%, 2% and 3%) to RAW cell cultured in the presence of RANKL increased the number of osteoclast up to 1500% with 2% of sodium pyruvate and compared to the positive control (culture medium with RANKL). RAW cells cultures for 7 days in the presence of a combination of RANKL, 50 μg/ml ascorbic acid and 1% sodium pyruvate formed 2400% of TRAP positive cells compared to the positive control (culture medium with RANKL).

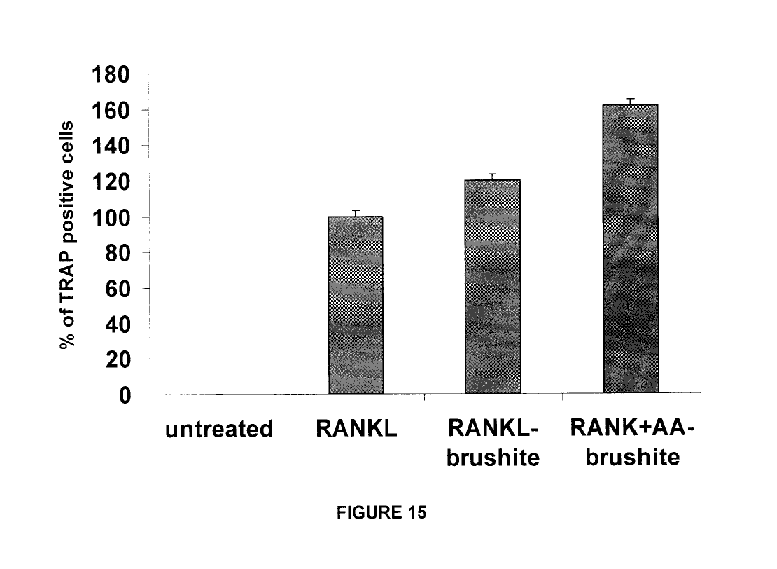

We investigated the capability of brushite cement cylinder(s) loaded with RANKL (800 ng) and ascorbic acid (800 μg) to induce osteoclastogenesis. RAW 264.7 cells were cultured during 7 days in the presence of brushite cement cylinders either loaded with RANKL (800 ng) alone or with RANKL (800 ng) and ascorbic acid (800 μg). Osteoclast formation was increased by 60% and 40% compared to positive control (culture medium with RANKL) and compared to RAW cell cultured in the presence of RANKL-coated brushite cement, respectively (FIG. 15).

### Other Embodiments

While specific embodiments have been described, those skilled in the art will recognize many alterations that could be made within the spirit of the invention, which is defined solely according to the following claims:

1. A matrix for inducing or enhancing bone formation, the matrix comprising: a material having an osteoclastogenic agent associated therewith, the agent being releasable from the material in an amount which is sufficient to induce or enhance one or both of osteoclast differentiation and bone formation.

2-7. (canceled)

8. The matrix, according to claim 1, in which the material is selected from one or more of the group consisting of a biomaterial, a ceramic, a non-metallic coating, a calcium phosphate-containing cement, a cement reactant, a mixture of cement reactants, a cement reactant in a slurry, a diphosphate, a triphosphate, a tetraphosphate, a polyphosphate, hydroxyapatite, brushite, tricalcium phosphate, monetite, tetracalcium phosphate, calcium silicate, calcium sulphate, calcium carbonate, magnesium phosphate, magnesium carbonate, magnesium sulphate, a hydrogel, a polymer and a metal.

9-16. (canceled)

17. The matrix, according to claim 1, in which the osteoclastogenic agent is at a concentration selected from the group consisting of
a) 1 ng or more and 1000000 ng or less of agent per mg of material;
b) 10 ng or more and 500 ng or less of agent per mg of material; and
c) 15 ng or more and 25 ng or less of agent per mg of material.

18. (canceled)

19. (canceled)

20. The matrix, according to claim 1, in which the osteoclastogenic agent is RANKL.

21. The matrix, according to claim 20, in which the RANKL is in combination another one or more components selected from the group consisting of:
a) an antioxidant;
b) an antioxidant and brushite;
c) an antioxidant and hydroxyapatite,
d) pyruvate salts or pyruvic acid; and
e) pyruvate salts, pyruvic acid, ascorbic acid, or trehalose.

22. (canceled)

23. (canceled)

24. The matrix, according to claim 21, in which the antioxidant is ascorbic acid or dehydroascorbic acid or salts thereof.

25-29. (canceled)

30. The matrix, according to claim 20, in which the RANKL is stable at ambient temperature and up to 37° C.

31. (canceled)

32. (canceled)

33. The matrix, according to claim 1, in which the osteoclastogenic agent is in an amount sufficient to cause or enhance implant osteointegration, remodelling of the matrix, fracture union or defect repair.

34. (canceled)

35. A composition for promoting osteoclastogenesis in vitro or in vivo, the composition comprising: a combination of RANKL and an agent to prolong RANKL stability.

36-41. (canceled)

42. A composition for inducing differentiation or tissue repair in vitro or in vivo, the composition comprising: compound for enhancing the bioactivity of an inductive protein.

43. The composition, according to claim 42, in which the compound is selected from one or more of the group consisting of pyruvate, a product or substrate of glycolisis, a product or substrate of the citric acid cycle, lactate, oxaloacetate, malate, succinate, a ketone, a keto-acid, glucose α-ketoglutarate, and citrate or is any compound that may react to form or be metabolised to form one or more of the group consisting of a pyruvate, a product or substrate of glycolisis, a product or substrate of the citric acid cycle, lactate, oxaloacetate, malate, succinate, a ketone, a keto-acid, glucose α-ketoglutarate, and citrate.

44. The composition, according to claim 42, in which the inductive protein is RANKL.

45. The composition, according to claim 44, in which the pyruvate and the RANKL are in a matrix or are in solution.

46. (canceled)

47. The composition, according to claim 45, in which the pyruvate and the RANKL are in combination with ascorbic acid.

48. A method of promoting osteoclastogenesis in vitro or in vivo, the method comprising: differentiating progenitor cells into osteoclasts in contact with, within or localized near the composition, according to claim 35.

49. The method, according to claim 48, in which the progenitor cells are osteoclast precursor cells.

50-57. (canceled)

58. A method of treating bone trauma in a subject, the method, comprising: implanting a matrix, according to claim 1, adjacent to or within a site of the trauma so as to enhance bone formation adjacent to or within a site requiring repair.

59. The method, according to claim 58, in which the bone trauma results from a bone degenerative disease.

60. The method, according to claim 58, in which the bone trauma results from surgery.

61. (canceled)

62. The method, according to claim 58, in which the bone trauma is a bone fracture.

63-68. (canceled)
